(19) United States
(12) Patent Application Publication
(10) Pub. No.: US 2026/0325850 A1
(43) Pub. Date: Oct. 22, 2026

(54) DELIVERY OF ANTI-TACHYCARDIA PACING THERAPY

(71) Applicant: Medtronic, Inc., Minneapolis, MN (US)

(72) Inventors: Troy E. Jackson, Rogers, MN (US); Wade M. Demmer, Coon Rapids, MN (US)

(21) Appl. No.: 19/114,969

(22) PCT Filed: Sep. 18, 2023
(86) PCT No.: PCT/IB2023/059235

§ 371 Date: Mar. 25, 2025

Related U.S. Application Data
(60) Provisional application No. 63/377,903, filed on Sep. 30, 2022.

(51) Int. Cl. A61N 1/365; A61N 1/02; A61N 1/372

(52) U.S. Cl. CPC A61N 1/36592; A61N 1/025; A61N 1/36585; A61N 1/37247; A61N 1/37252

(57) ABSTRACT

A system including an implantable medical device (IMD) configured to deliver an anti-tachycardia pacing (ATP) therapy to a patient and an external device including: communications circuitry configured to communicate with the IMD; and processing circuitry configured to: receive, via the communications circuitry, a request to connect from the IMD, determine whether the IMD is connected to the external device, and based on a determination that the IMD is connected to the external device, transmit instructions, via the communications circuitry, to the IMD to deliver the ATP therapy to the patient.

This application claims the benefit of U.S. Provisional Patent Application Ser. No. 63/377,903, filed 30 Sep. 2022, the entire content of which is incorporated herein by reference.

### TECHNICAL FIELD

The disclosure relates to medical devices, and, more particularly, to implantable medical devices configured to detect and treat cardiac arrhythmias with anti-tachycardia pacing (ATP) therapy.

### BACKGROUND

Implantable medical devices (e.g., implantable cardiac devices) may provide electrical overdrive stimulation, e.g., ATP therapy, to suppress or convert tachyarrhythmias, thereby supplying relief from symptoms and preventing or terminating arrhythmias that could lead to sudden cardiac death.

### SUMMARY

An implantable medical device (IMD) may deliver anti-tachycardia pacing (ATP) therapy to a heart of a patient to suppress or convert tachyarrhythmias (e.g., ventricular tachycardia (VT)). ATP therapy may provide the patient relief from symptoms and/or terminate arrhythmias experienced by the patient. Some IMDs may include pacing functionality and may be configured to deliver anti-tachyarrhythmia shocks to the patient instead of or in addition to the ATP therapy. Other IMDs may only be configured to deliver ATP therapy to the patient.

In some examples, delivery of the ATP therapy may produce unintended results within the patient. The unintended results may include, but are not limited to, acceleration of a hemodynamically-stable VT into a hemodynamically-unstable VT, acceleration into ventricular fibrillation (VF) or the like. Anti-tachyarrhythmia shocks and/or other electrical stimulation may need to be delivered to the patient to mitigate, terminate, and/or reverse the unintended results. Thus, a patient with an IMD only configured to deliver ATP therapy may want to have access to caretakers and/or medical care (e.g., emergency medical services (EMS)) to reduce the likelihood and/or severity of the unintended results.

In some examples, the techniques of this disclosure include connecting an IMD to an external device and/or user prior to delivery of the ATP therapy to the patient. The connection between the IMD and the external device may facilitate detection of any unintended results of the ATP therapy and the ability to provide emergency aid to the patient in the event of an unintended result.

In some examples, the disclosure describes selecting and/or adjusting the ATP therapy based on the availability of emergency aid. A medical device system may select the ATP therapy and/or adjust parameters of the ATP therapy based on the availability of the emergency aid to reduce a risk of an unintended result, e.g., if emergency aid is relatively unavailable.

In one example, this disclosure is directed to an implantable medical device (IMD) configured to deliver anti-tachycardia pacing (ATP) therapy to a patient, the IMD including: therapy delivery circuitry connected to one or more electrodes; communications circuitry; and processing circuitry configured to: transmit, via the communications circuitry, a request to connect to an external device, determine, based on received signals from the external device, whether the IMD is in communication with the external device; and based on a determination that the IMD is in communication with the external device, transmit instructions to the therapy delivery circuitry to deliver the ATP therapy to the patient.

In another example, this disclosure is directed to a system including: an implantable medical device (IMD) configured to deliver an anti-tachycardia pacing (ATP) therapy to a patient; and an external device including: communications circuitry configured to communicate with the IMD; and processing circuitry configured to: receive, via the communications circuitry, a request to connect from the IMD; determine whether the IMD is in communication with the external device; and based on a determination that the IMD is in communication with the external device, transmit instructions, via the communications circuitry, to the IMD to deliver the ATP therapy to the patient.

In another example, this disclosure is directed to a system including: an external device configured to communicate with a network; and an implantable medical device (IMD) including: memory configured to store a plurality of medical therapies, wherein each of the plurality of medical therapies is configured to be delivered by the IMD to a patient; communications circuitry configured to communicate with the external device; signal generation circuitry; and processing circuitry configured to: select a first medical therapy from the plurality of medical therapies; receive, from the external device and via the communications circuitry, data on one or more context factors for the patient; determine, based on the received data, whether the received data satisfies one or more threshold risk conditions of the patient; select, based on a determination that the received data satisfies at least one of the one or more threshold risk conditions of the first medical therapy, a second medical therapy from the plurality of medical therapies, wherein the second medical therapy is less aggressive than the first medical therapy; and deliver, via the signal generation circuitry, the second medical therapy to the patient.

In another example, this disclosure is directed to a method including: transmitting, by communications circuitry of an implantable medical device (IMD), a request to connect to an external device; receiving, by the communications circuitry, signals from the external device; determining, by processing circuitry of the IMD and based on the received signals, whether the IMD is in communication with the external device; and based on a determination that the IMD is in communication with the external device, delivering, by therapy delivery circuitry of the IMD, anti-tachycardia pacing (ATP) therapy to a patient.

In another example, this disclosure is directed to a method including: receiving, by processing circuitry of an external device, a request to connect from an implantable medical device (IMD); determining, by the processing circuitry, whether the IMD is in communication with the external device; and transmitting, by the processing circuitry and based on a determination that the IMD is in communication with the external device, instructions to the IMD to deliver an anti-tachycardia pacing (ATP) therapy to a patient.

In another example, this disclosure is directed to a method including: selecting, by processing circuitry of an IMD, a first medical therapy from a plurality of medical therapies, wherein each of the plurality of medical therapies is configured to be delivered by the IMD to a patient; receiving, from a network and via an external device, data on one or more context factors for the patient; determining, by the processing circuitry and based on the received data, whether the received data satisfies one or more threshold risk conditions of the patient; selecting, by the processing circuitry and based on a determination that the received data satisfies at least one of the one or more threshold risk conditions of the first medical therapy, a second medical therapy from the plurality of medical therapies, wherein the second medical therapy is less aggressive than the first medical therapy; and delivering, by signal generation circuitry of the IMD, the second medical therapy to the patient.

In another example, this disclosure is directed to a computer readable storage medium including instructions that, when executed, cause processing circuitry within a system to perform a method including: causing communications circuitry of an implantable medical device (IMD) to transmit a request to connect to an external device; receiving, via the communications circuitry, signals from the external device; determining, base don the received signals, whether the IMD is in communication with the external device; and based on a determination that the IMD is in communication with the external device, causing therapy delivery circuitry of the IMD to deliver anti-tachycardia pacing (ATP) therapy to a patient.

In another example, this disclosure is directed to a computer readable storage medium including instructions that, when executed, cause processing circuitry within a system to perform a method including: receiving a request to connect from an implantable medical device (IMD), determine whether the IMD is in communication with the external device; and transmit, based on a determination that the IMD is in communication with the external device, instructions to the IMD to deliver an anti-tachycardia pacing (ATP) therapy to a patient.

In another example, this disclosure is directed to a computer readable storage medium including instructions that, when executed, cause processing circuitry within a system to perform a method including; selecting a first medical therapy from a plurality of medical therapies, wherein each of the plurality of medical therapies is configured to be delivered by the IMD to a patient; receiving, from a network and via an external device, data on one or more context factors for the patient; determining, by the processing circuitry and based on the received data, whether the received data satisfies one or more threshold risk conditions of the patient; selecting, based on a determination that the received data satisfies at least one of the one or more threshold risk conditions of the first medical therapy, a second medical therapy from the plurality of medical therapies, wherein the second medical therapy is less aggressive than the first medical therapy; and causing signal generation circuitry to deliver the second medical therapy to the patient.

In another example, this disclosure is directed to a system including an IMD and an external device. The IMD is configured to deliver anti-tachycardia pacing (ATP) therapy to a patient and includes: therapy delivery circuitry connected to one or more electrodes; communications circuitry; and processing circuitry configured to transmit, via the communications circuitry, a request to connect to the external device; determine, based on received signals from the external device, whether the IMD is in communication with the external device; and based on a determination that the IMD is in communication with the external device, transmit instructions to the therapy delivery circuitry to deliver the ATP therapy to the patient. The external device includes: communications circuitry configured to communicate with the IMD; and processing circuitry configured to: receive, via the communications circuitry, the request to connect from the IMD; determine whether the IMD is in communication with the external device; and based on a determination that the IMD is in communications with the external device, transmit instructions, via the communications circuitry, to the IMD to delivery the ATP therapy to the patient.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

FIG. 1 is a conceptual drawing illustrating an example medical device system, in accordance with one or more aspects of this disclosure.

FIG. 2 is a functional block diagram illustrating an example configuration of the IMD of FIG. 1.

FIG. 3 is a functional block diagram illustrating an example configuration of the external device of FIG. 1.

FIG. 4 is a flowchart illustrating an example process of delivering medical therapy to the patient.

FIG. 5 is a flowchart illustrating another example process of delivering medical therapy to the patient.

### DETAILED DESCRIPTION

An implantable medical device (IMD) may deliver anti-tachycardia pacing (ATP) therapy to a heart of a patient to suppress or convert tachyarrhythmias (e.g., ventricular tachycardia (VT)). The IMD may sense signals from the heart of the patient, determining that the patient is experiencing tachyarrhythmia, and deliver ATP therapy to the patient. In some examples, delivery of the ATP therapy may produce unintended results within the patient. The unintended results may include, but are not limited to, acceleration of a hemodynamically-stable VT into a hemodynamically-unstable VT, acceleration into ventricular fibrillation (VF) or the like. Anti-tachyarrhythmia shocks and/or other electrical stimulation may need to be delivered to the patient to mitigate, terminate, and/or reverse the unintended results. In some examples, the IMD may be incapable of delivering the anti-tachyarrhythmia shocks or other electrical stimulation to address the unintended results

The devices, systems, and methods described in the disclosure may increase the likelihood of addressing the unintended results and/or reducing a likelihood of an occurrence of the unintended results. An IMD described in this disclosure may request connection with an external device and/or system (e.g., a patient monitoring system, an EMS system, or the like) and/or a user (e.g., EMS, a healthcare provider, a family member, or the like) and may confirm the connection with the external device before delivering ATP therapy to the patient. If the patient experienced an unintended result, the connection between the IMD and the external device may facilitate notification of the occurrence of the unintended result to users capable of delivering the anti-tachyarrhythmia shocks and may reduce a time it takes between the occurrence of the unintended result and the delivery of the anti-tachyarrhythmia shocks.

In some examples, certain context factors of a scenario surrounding a patient may increase and/or decrease the risk of an occurrence of an unintended result and/or increase and/or decrease the severity of any occurrences of unintended results. The factors may include, but are not limited to, a distance of any medical service providers from the patient, a response time of any nearby medical service providers, health of the patient, or behavioral patterns of the patient. In such examples, the devices, systems, and methods described in the disclosure may select, based on data corresponding to the factors, a relatively less aggressive ATP therapy (e.g., a less proarrhythmic ATP therapy) or a relatively more aggressive ATP therapy (e.g., a more proarrhythmic ATP therapy). The use of context factors and other data (e.g., behavioral data, clinical data, or the like), may provide the patient with a more efficient delivery of medical therapy, e.g., by reducing the risk of an occurrence of an unintended result while delivering an optimal ATP therapy to the patient.

While the devices, systems, and methods of the disclosure are described primarily with reference to the delivery of ATP therapy to treat tachyarrhythmia, the devices, systems, and methods may also be used for other medical therapies delivered to patients to treat other medical conditions.

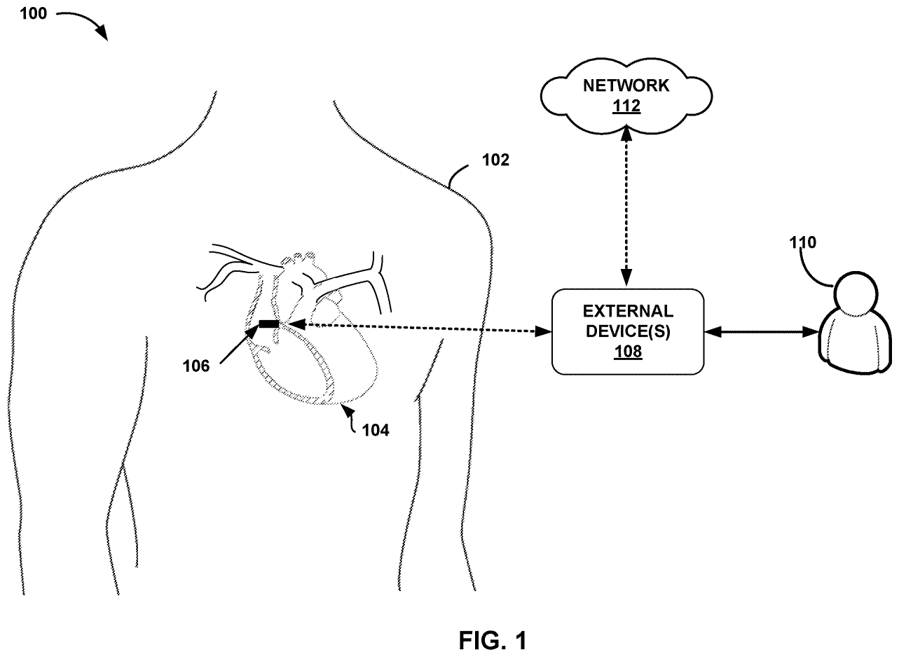

FIG. 1 is a conceptual drawing illustrating an example medical device system 100, in accordance with one or more aspects of this disclosure. Medical device system 100 includes an implantable medical device (IMD) 106 implanted within a heart 104 of a patient 102. IMD 106 communicates with one or more external devices 108. Each of external devices 108 may communicate with network 112 and/or user 110.

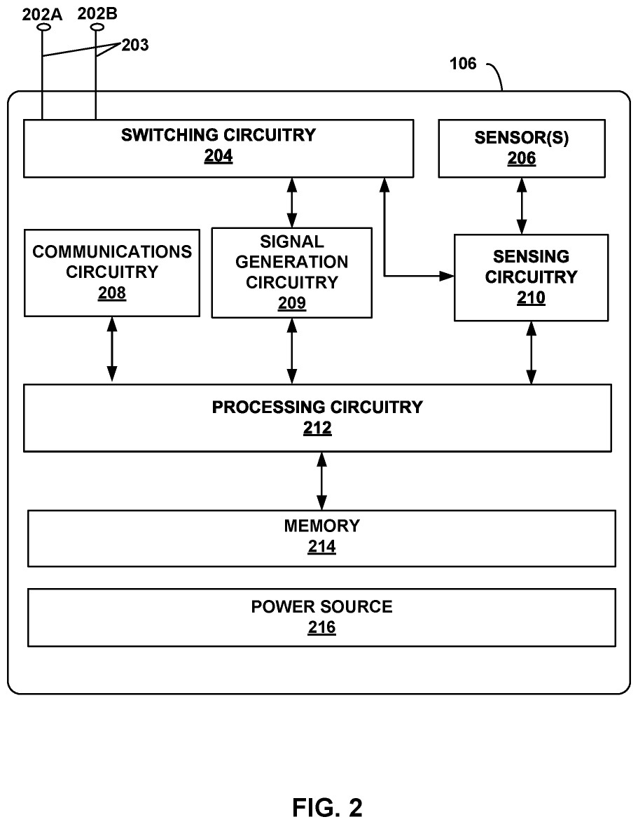

IMD 106 may sense signals from heart 104, determine the occurrence of tachyarrhythmia within heart 104, and deliver ATP therapy to heart 104. In some examples, as illustrated in FIG. 1, IMD 106 may be wholly implanted within heart 104 (e.g., within one or more chambers of heart 104) and may deliver sense signals from heart 104 and/or deliver ATP therapy and/or other electrical stimulation signals to heart 104 via one or more electrodes (not pictured). In some examples, at least a portion of IMD 106 (e.g., a housing of IMD 106, as described in further detail with respected to FIG. 2) may be implanted outside of heart 104 and within patient 102 (e.g., within vasculature of patient 102, within a subcutaneous pocket within patient 102, or the like). One or more electrodes of IMD 106 may be connected to tissue of heart 104 (e.g., wall tissue of heart 104) and may be connected to the portion of IMD 106 outside of heart 104, e.g., via one or more electrical leads.

IMD 106 may detect and record heart rate data from heart 104. The heart rate data may include, but is not limited to rates/intervals determined based on detecting depolarizations of one or more chambers (e.g., left ventricle (LV), right ventricle (RV), left atrium (LA), right atrium (RA)) of heart 104. IMD 106 may determine a heart rate (e.g., a ventricular rate) of heart 104 and determine if the heart 104 of patient 102 is operating in a manner that may correspond to tachyarrhythmia. For example, IMD 106 determines that patient 102 may be experiencing tachyarrhythmia based on one or more electrical signals (e.g., heart rate, irregularity of rhythm of heart 104, electrical morphology of signals of heart 104) and/or non-electrical signals (e.g., pressure, acceleration, or the like) detected by IMD 106.

IMD 106 may be in wireless communications with one or more external devices 108. External devices 108 may include computing devices configured to monitor a status of IMD 106 or of patient 102. External devices 108 may include computing devices including, but not limited to, tablets, smartphones, smartwatches, laptop computers, or desktop computers. In some examples, an external device 108 may be a part of a medical care systems (e.g., an EMS system, a computing system of a hospital, or the like). External device 108 may further include, but is not limited to, a wearable defibrillator, or an automated external defibrillator (AED). In some examples, external device 108 may include displays and/or user interfaces (UIs) which are interactable by a user 110. User 110 may include family members, medical care providers, EMS providers, and/or any other persons capable of delivering anti-tachyarrhythmia shocks to patient 102. External device 108 may wirelessly communicate with IMD 106 and/or network 112 according to one or more wireless communications protocols (e.g., according to the Bluetooth® or Bluetooth® Low Energy (BLE) protocols).

In some examples, when external device 108 is an AED or a wearable defibrillator, external device 18 may, in response to a notification from IMD 106 (e.g., indicating the presence of tachyarrhythmia, a need to deliver ATP pacing, the presence of one or more adverse events, or the like), notify user 110 to bring external device 108 to patient 102, e.g., to deliver electrical therapy signals to user 110. External device 108 may output auditory, visual, tactile, or other outputs to alert user 110 to bring external device 108 to patient 102 and/or to alert user 110 of the position of external device 108.

Network 112 may communicate with external device 108 and transfer data between network 112 and external device 108. In some examples, behavioral data, clinical data, and/or data on one or more context factors (e.g., distance of patient 102 from a medical care provider, a response time of a medical care provider, or the like). The data may be stored on one or more computing devices, computing systems, and/or cloud computing environments in communication with network 112.

Network 112 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 112 may include one or more networks administered by service providers, and may thus form part of a large-scale public network infrastructure, e.g., the Internet.

In some examples, prior to delivery of ATP therapy to heart 104, IMD 106 may transmit a request to connect to external device 108. For example, IMD 106 may transmit one or more advertisements for a wireless connection. External device 108 may transmit a signal to IMD 106 indicating that external device 108 is connected to IMD 106. For example, external device 108 may transmit a response to the advertisements, and IMD 106 and external device may exchange one or more messages to establish a communication session. The signal may be the response or another message during establishing the communication session, or a dedicated message transmitted by external device 108 after the session is established. IMD 106 may receive the signal from external device 108 and deliver ATP therapy to heart 104 upon confirmation that IMD 106 is connected to external device 108.

In some examples, external device 108 may retrieve data (e.g., data on context factors, behavioral data, clinical data, or the like) from network 112 and/or IMD 106. Based on the retrieved data, external device 108 may select an appropriate ATP therapy and/or one or more parameters of an ATP therapy and transmit instructions to IMD 106 to deliver the select ATP therapy to heart 104. The selected ATP therapy may reduce a likelihood of an occurrence of an unintended result while suppressing tachyarrhythmia experienced by patient 102.

FIG. 2 is a functional block diagram illustrating an example configuration of IMD 106 of FIG. 1. IMD 106 may be an implanted cardiac device including, but is not limited to, an implantable pacing device, an implantable pulse generator (IPG), an implantable cardioverter defibrillator (ICD), a cardiac resynchronization therapy (CRT) device, or the like. IMD 106 may include components including, but is not limited to, electrodes 202A-B (hereinafter referred to as “electrodes 202”), switching circuitry 204, sensor(s) 206, communications circuitry 208, signal generation circuitry 208, sensing circuitry 210, processing circuitry 212, memory 214, and a power source 216 that provides operational power to the other components. The various circuitry may be, or include, programmable or fixed function circuitry configured to perform the functions attribute to respective circuitry.

Memory 214 may store computer-readable instructions that, when executed by processing circuitry 212, cause IMD 106 to perform various functions. Memory 214 may be a storage device or other non-transitory medium. Memory 214 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Electrodes 202 are electrically connected to wall tissue of one or more chambers of heart 104, e.g., via at least one of electrodes 202 being in physical contact with the wall tissue. Electrodes 202 may be electrically connected to switching circuitry 204 of IMD 106 through electrical connectors 203. In some examples, where a portion of IMD 106 including switching circuitry 204 is implanted outside of heart 104, electrical connectors 203 may include electrical leads extending into heart 104 and connecting electrodes 202 to the wall tissue of heart 104. In other examples, electrodes 202 may be directly disposed on IMD 106, e.g., on a housing of IMD 106. Multiple electrodes of electrodes 202 may be connected to a same chamber of heart 104 or to different chambers of heart 104. While IMD 106 illustrated in FIG. 2 includes two electrodes 202, other example IMDs may include three or more electrodes 202.

Switching circuitry 204 may selectively couple sensing circuitry 210 and/or signal generation circuitry 209 to selected combinations of electrodes 202, e.g., to sense the electrical activity of the atria and/or the ventricles of heart 104 or to deliver electrical stimulation signals (e.g., of an ATP therapy) to wall tissue of heart 104, respectively. Switching circuitry 302 may include one or more switch arrays, one or more multiplexers, one or more switches (e.g., a switch matrix or other collection of switches), one or more transistors, or other electrical circuitry.

Sensing circuitry 210 may include filters, amplifiers, analog-to-digital converters, or other circuitry configured to sense cardiac electrical signals via electrodes 202 or to deliver electrical signals to cardiac tissue via electrodes 202. In some examples, sensing circuitry 210 is configured to detect events, e.g., depolarizations, within the cardiac electrical signals and provide indications thereof to processing circuitry 212. In this manner, processing circuitry 212 may determine heart rate and/or ventricular rate of heart 104 based on the sensed cardiac electrical signals and may determine whether patient 102 may be experiencing tachyarrhythmia based on the determined heart rate and/or ventricular rate.

Sensing circuitry 210 may be electrically connected to one or more sensors (not pictured) disposed within IMD 106. The one or more sensors may be configured to sense other signals (e.g., mechanical signals, chemical signals, non-cardiac electrical signals, or the like) and processing circuitry 212 may determine heart rate, ventricular rate, and/or presence of tachyarrhythmia based at least in part on the sensed signals. The one or more sensors may include, but are not limited, an accelerometer, an oxygenation sensor, a chemical sensor, or the like. Processing circuitry 212 may store the sensed cardiac electrical signals, the determined heart rate, sensed mechanical signals and/or the determined ventricular rate in memory 214.

Processing circuitry 212 may include any one or more of a microprocessor, a controller, a digital signal processor (DSP), an application-specific integrated circuit (ASIC), a field-programmable gate array (FPGA), discrete logic circuitry, or any other processing circuitry configured to provide the functions attributed to processing circuitry 212 herein and may be embodied as firmware, hardware, software, or any combination thereof.

Processing circuitry 212 may determine the heart rate, e.g., ventricular rate of heart 104, based on the sensed data (e.g., sensed electrical signals from electrodes 202, sensor data from sensor(s) 204). Processing circuitry 212 may determine whether patient 102 may be experiencing tachyarrhythmia based on the determined heart rate. In some examples, processing circuitry 212 may determine whether patient 102 is experiencing one or more other cardiac conditions, e.g., one or more other types of tachycardia.

Processing circuitry 212 may, after determining patient 102 may be experiencing tachyarrhythmia, instruct communications circuitry 208 to send a request to connect to external device 108. After communications circuitry 208 receives confirmation of connection of IMD 106 to external device 108 (i.e., IMD 106 is in communication with external device 108), processing circuitry 212 may then instruct signal generation circuitry 209 to generate and deliver an electrical stimulation signal (e.g., ATP therapy) to cardiac tissue of heart 104 via electrodes 202 and switching circuitry 204. If communications circuitry 208 does not receive confirmation of the connection to external device 108, processing circuitry 212 may instruct communications circuitry 208 to re-send the request to connect after a period of time. A clinician may adjust the period of time, e.g., based on the medical needs of patient 102. In some examples, processing circuitry 212 does not deliver any electrical stimulation signals to heart 104 unless processing circuitry 212 receives the confirmation of connection to external device 108.

Processing circuitry 212 may determine, based at least in part on the sensed data, that patient 102 is experiencing one or more adverse event. The adverse events may include one or more conditions experienced by patient 102 which may indicate to IMD 106 that patient 102 is experiencing hemodynamic collapse. The adverse events may include, but are not limited to, a fall of patient 102, a cessation of breathing by patient 102, a more serious (e.g., lethal) tachycardiac event experienced by patient 102, a motion of heart 104 (e.g., an increase in motion, a cessation of motion, a decrease in motion) of patient 102, a drop in tissue oxygenation of patient 102, or the like. If processing circuitry 212 determines the occurrence of at least one adverse event, processing circuitry 212 may instruct signal generation circuitry 209 to deliver electrical stimulation signals to heart 104 without requiring that IMD 106 be connected to external device 108.

In some examples, e.g., after processing circuitry 212 receives confirmation that IMD 106 is connected to external device 108, processing circuitry 212 may receive an ATP therapy and/or parameters of an ATP therapy to be delivered to heart 104, e.g., from external device 108. The parameters may include ATP parameters and/or a number of safety checks performed by IMD 106 prior to delivery of the electrical stimulation signal. IMD 106 performs the safety checks to determine whether IMD 106 is detecting an arrhythmia that requires delivery of the electrical stimulation signal to heart 104. Safety checks may include, but are not limited to, determining whether patient 102 is exercising or engaging in strenuous activity or determining whether patient 102 is in an appropriate location (e.g., near an AED, near a caregiver, or the like). Processing circuitry 212 may adjust the therapy configured to be delivered by signal generation circuitry 209 (e.g., to conform with the received ATP therapy and/or the received parameters) and instruct signal generation circuitry 209 to deliver electrical stimulation signals corresponding to the adjusted ATP therapy and/or the adjusted parameters to heart 104.

ATP therapy parameters may include pulse intervals, pulse width, current and/or voltage amplitudes, and durations for each pacing mode. For example, the pulse interval may be based on a fraction of the detected ventricular tachycardia (VT) cycle length and be between approximately 150 milliseconds (ms) and 500 ms (e.g., between approximately 2.0 hertz (Hz) and 7.0 Hz), and the pulse width may be between approximately 0.5 ms and 2.0 ms. The amplitude of each pacing pulse may be between approximately 2.0 volts (V) and 10.0 V. In some examples, the pulse amplitude may be approximately 6.0 V and the pulse width may be approximately 1.5 ms; another example may include pulse amplitudes of approximately 5.0 V and pulse widths of approximately 1.0 ms. Each train of pulses during ATP therapy may last for a duration of between approximately 0.5 seconds(s) to approximately 15 s or be defined as a specific number of pulses. Each pulse, or burst of pulses, may include a ramp up in amplitude or in pulse rate. In addition, trains of pulses in successive ATP periods may be delivered at an increasing pulse rate to capture heart 104 and terminate the tachyarrhythmia.

Signal generation circuitry 209 may be configured to generate electrical stimulation signals (e.g., of an ATP therapy) and deliver the generated electrical stimulation signals to heart 104 through electrodes 202, conductors 203, and switching circuitry 204. Signal generation circuitry 209 may include, as examples, current or voltage sources, capacitors, charge pumps, or other signal generation circuitry.

Sensor(s) 206 may include one or more sensing elements that transduce patient physiological activity to an electrical signal to sense values of a respective patient parameter. Sensor(s) 206 may include one or more accelerometers, optical sensors, chemical sensors, temperature sensors, pressure sensors, or any other types of sensors. Sensor(s) 206 may output patient parameter values that may be used by processing circuitry 212 to determine the heart rate, the ventricular rate, and/or the occurrence of one or more of the adverse events.

Communications circuitry 208 (alternatively referred to as “telemetry circuitry 312”) supports wireless communication between IMD 106 and external device 108. Processing circuitry 212 of IMD 106 may transmit to external device 108 a request to connect via communications circuitry 312. Processing circuitry 212 may receive, via communications circuitry 312, a confirmation of a connection between IMD 106 and external device 108. Processing circuitry 312 may transmit, to external device 108 and via communications circuitry 312, sensed data and receive, from external device 108 and via communications circuitry 312, adjusted ATP therapy and/or adjusted parameters of an ATP therapy, e.g., based at least in part on the sensed data.

Communications circuitry 208 may communicate with external device 108 by wireless communication techniques. Wireless communication techniques may include radiofrequency (RF) communication techniques, e.g., via an antenna (not shown). In some examples, system 100 may include a second IMD is communication with IMD 106. IMD 106 may communicate with the second IMD via communications circuitry 208 and second IMD may relay the communications from IMD 106 to external device 108, e.g., via wireless communication techniques. In some examples, the second IMD may, after receiving communications from IMD 106, determine whether the decisions made by IMD 106 are accurate. If the second IMD determines that the decisions (e.g., determination of the presence of tachyarrhythmia, decisions to delivery electrical stimulation signals to heart 104, or the like) made by IMD 106 are accurate, the second IMD may continue to relay the decisions to external device 108. If the second IMD determines that the decisions made by IMD 106 are inaccurate, the second IMD may adjust and/or overrule the decisions made by IMD 106. In some examples, instead of or in addition to a second IMD, IMD 106 may similarly communicate with a wearable medical device, such as a patch, watch, other wrist-worn device, or garment.

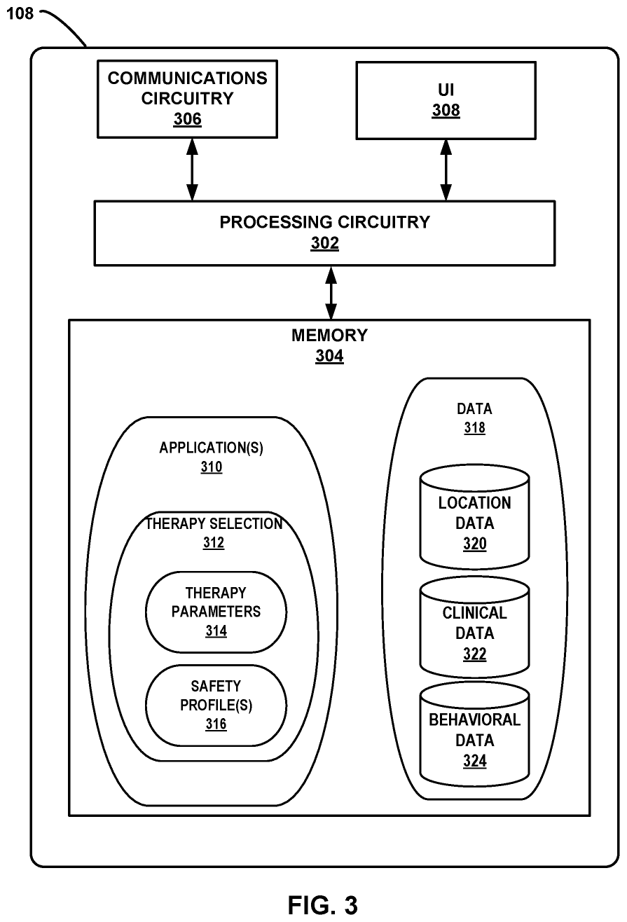

FIG. 3 is a functional block diagram illustrating an example configuration of external device 108 of FIG. 1. As shown in FIG. 3, external device 108 may include processing circuitry 302, memory 304, communications circuitry 306, and user interface (UI) 308. Memory 304 may include one or more modules including application(s) module 310 and data module 318. Application(s) module 310 may include therapy selection module 312 which includes therapy parameters module 314 and safety profile(s) module 316. Data 318 stored in memory 304 may include location data 320, clinical data 322, and behavioral data 324.

Processing circuitry 302 may include fixed function circuitry and/or programmable processing circuitry. Processing circuitry 302 may include any one or more of a microprocessor, a controller, a GPU, a TPU, a digital signal processor (DSP), an ASIC, a FPGA, or equivalent discrete or analog logic circuitry. In some examples, processing circuitry 302 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 302 herein may be embodied as software, firmware, hardware, or any combination thereof. In some examples, memory 304 includes computer-readable instructions that, when executed by processing circuitry 302, cause external device 108 and processing circuitry 302 to perform various functions and/or processes attributed herein to external device 108, network 112, and/or processing circuitry 302. Memory 304 may include any volatile, non-volatile, magnetic, optical, or electrical media, such as RAM, ROM, NVRAM, EEPROM, flash memory, or any other digital media.

Processing circuitry 302 may receive, via communications circuitry 306, a request to connect from IMD 106. Processing circuitry 302 may transmit to IMD 106, via communications circuitry 306, a confirmation of a connection after receiving the request to connect. In some examples, processing circuitry 302 may determine that user 110 is connected to external device 108 and confirm the connection between IMD 106 and external device 108 based on a determination that user 110 is connected to external device 108.

In some examples, where user 110 is one or more other persons (e.g., a medical care provider, a family member, or the like), processing circuitry 302 may query user 110 via UI 308 and determine whether external device 108 is accessible to user 110 (e.g., whether user 110 is using or has access to external device 108) based on user feedback, or lack thereof. UI 308 may receive the user feedback and transmit the user feedback to processing circuitry 302, e.g., in the form of electronic communications. In some examples, where external device 108 is a wearable device worn by user 110 (e.g., a smartwatch or the like), UI 308 may determine whether external device 108 is accessible to user 110 by determining whether user 110 is physically connected to (e.g., wearing) and/or in physical contact with external device 108 by detecting the pulse of user 110. In some examples, where user 110 is a computing system, computing device, cloud computing environment, or the like (e.g., an EMS server), processing circuitry 302 may transmit a request to connect to user 110 via communications circuitry 306 and determine the presence of a connection upon reception of a confirmation of the connection from user 110.

Processing circuitry 302 may retrieve and execute instructions stored in therapy selection module 312 of application(s) module 310 of memory 304 to select an ATP therapy stored in therapy selection module 312. The ATP therapy may include parameters of the ATP therapy, stored in therapy parameters module 314, and a number of safety checks, stored in safety profile(s) module 316. In other examples, the information stored in therapy selection module 312 may be stored in network 112 and processing circuitry 302 may retrieve the information from network 112 via communications circuitry 306.

Processing circuitry 302 may retrieve, from memory 304 and/or network 112, data 318 for one or more context factors for patient 102. The one or more context factors may include, but are not limited to, availability (e.g., proximity, responsiveness) of a medical service provider, a medical care provider. and/or an EMS provider, health metrics (also referred to as “health status”) of patient 102 (e.g., age, weight, presence of co-morbidities, other medical conditions, or the like), or behavioral patterns of patient 102 (e.g., exercise patterns of patient 102, level of exercise of patient 102, or the like). In some examples, data corresponding to locations of patient 102, medical service providers, medical care providers, EMS, providers or the like may be stored in location data 320. A location of patient 102 may be programmed, or determined based on a location of IMD 106 and/or external device 108. Data corresponding to health metrics of patient 102 may be stored in clinical data 322. Data corresponding to behavioral patterns of patient 102 may be stored in behavioral data 324. In some examples, data 318 includes data obtained from network 112, user 110 (e.g., via UI 308), and/or IMD 106 (e.g., from sensed data from IMD 106).

Each of a plurality of ATP therapies stored in therapy selection module 312 may have a corresponding threshold risk condition for each of the one or more context factors. For example, one ATP therapy may have a maximum allowable distance between patient 102 and a medical service provider, medical care provider and/or an EMS provider and may have a maximum allowable response time between patient 102 and the medical service provider, medical care provider, and/or EMS provider.

Some of the plurality of ATP therapies may be more aggressive (e.g., more proarrhythmic) or less aggressive (e.g., less proarrhythmic) than another of the plurality of ATP therapies. More aggressive ATP therapies may require fewer safety checks, and/or may have higher maximum therapy amplitudes and/or therapy delivery frequencies than less aggressive ATP therapies. More aggressive ATP therapies may also have lower threshold risk conditions than less aggressive ATP therapies. For example, a more aggressive ATP therapy requires a lower maximum allowable distance, a lower maximum allowable response time, better health metrics of patient 102, and/or more consistent behavior of patient 102 than a less aggressive ATP therapy, e.g., due to an increased risk of an unintended event and/or increased severity of any unintended events. In another example, a less aggressive ATP therapy may require a higher maximum allowable distance, a higher maximum allowable response time, worse health metrics of patient 102, and/or less consistent behavior of patient 102, e.g., due to a decreased risk of an unintended event and/or a reduced severity of any unintended events. In some examples, behavior of patient 102 (e.g., exercising) may lead to potential false positive reports of tachyarrhythmia. In such cases, safety checks allow system 100 to detect the occurrence of false positives and prevent delivery of ATP therapy to heart 104 during such occurrences. Less aggressive ATP therapies may perform an increased number of safety checks and may be more likely to detect false positive occurrences, e.g., due to the increased number of safety checks.

In some examples, processing circuitry 302 may select a first ATP therapy and then replace the first ATP therapy with another ATP therapy based on data 318 on the one or more context factors. If data 318 satisfies at least one of the threshold risk conditions of the first ATP therapy, processing circuitry 302 may select a second, less aggressive ATP therapy where data 318 does not satisfy any of the threshold risk conditions of the second ATP therapy. In some examples, if data 318 does not satisfy any of the threshold risk conditions of the first ATP therapy, processing circuitry 302 may select a third, more aggressive ATP therapy instead, wherein data 318 does not satisfy any of the threshold risk conditions of the third ATP therapy but would satisfy at least one threshold risk condition of an ATP therapy more aggressive than the third ATP therapy.

Once processing circuitry 302 selects an ATP therapy, processing circuitry 302 may transmit, via communications circuitry 306, to IMD 106 deliver the selected ATP therapy to patient 102. In some examples, when IMD 106 is already configured to deliver an existing ATP therapy, processing circuitry 302 may instruct IMD 106 to replace the existing ATP therapy with the selected ATP therapy or to modify parameters of the existing ATP therapy to correspond to the selected ATP therapy.

Memory 304 may store information within external device 108, e.g., for processing during operation of external device 108. Memory 304 may be described as a computer-readable storage medium. In some examples, memory 304 includes temporary memory or a volatile memory including, but is not limited, random access memories (RAM), dynamic random access memories (DRAM), static random access memories (SRAM), or other forms of volatile memories known in the art. Memory 304, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable memories (EEPROM). In some examples, memory 304 includes cloud-associated storage.

Communications circuitry 306 may facilitate communication between processing circuitry 302 of external device 108 and IMD 106 and/or network 112. Communications circuitry 306 may communicate with other devices and/or systems via wired and/or wireless communication techniques. Wireless communications techniques may include RF communications techniques, e.g., via an antenna (not shown). Communications circuitry 306 may include a radio transceiver configured for communication according to standards of protocols, such as 3G, 4G, 5G, Wi-Fi (e.g., 802.11 or 802.15 ZigBee), Bluetooth®, or Bluetooth® Low Energy (BLE).

UI 308 may be configured to receive input, e.g., from patient 110 or another user. Examples of input are tactile, audio, kinetic, or optical input. UI 308 may include a mouse, a keyboard, voice responsive system, a camera, buttons a control pad, a microphone, a presence-sensitive or touch-sensitive component (e.g., a screen), or any other device for detecting input from user 110. UI 308 may also be configured to generate output, e.g., to patient 102 or another user. Examples of output include tactile, haptic, audio, or visual output.

UI 308 of external device 108 may include a presence-sensitive screen, a sound card, a video graphics adapter card, speakers, a cathode ray tube (CRT) monitor, a liquid crystal display (LCD), light emitting diodes (LEDs), or any other type of device for generating output to user 110.

In some examples, as illustrated in FIG. 3, application(s) 310 may be executed in a user space in external device 108. As a part of the execution of application(s) 310, processing circuitry 302 may select an ATP therapy, e.g., as described in greater detail above.

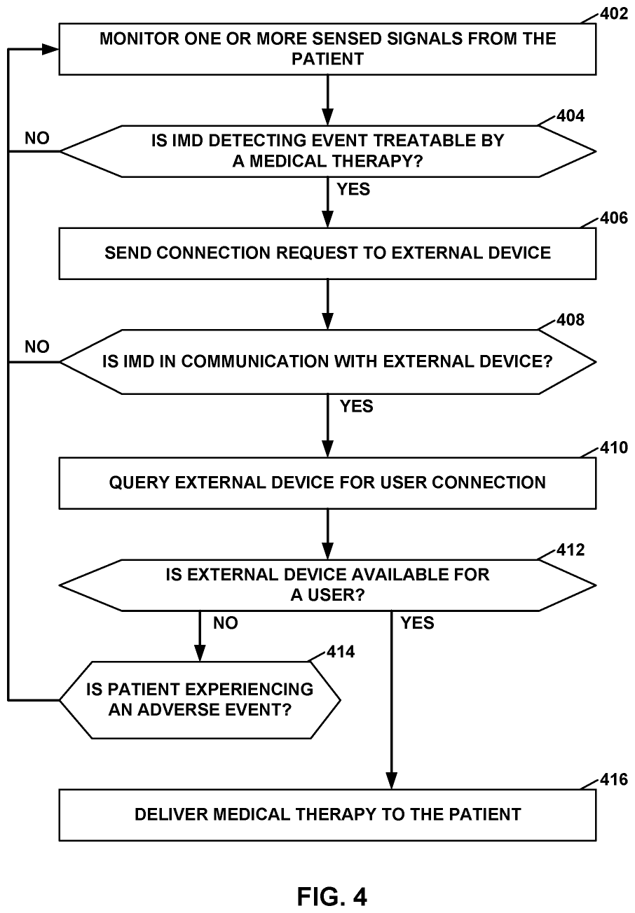

FIG. 4 is a flowchart illustrating an example process of delivering medical therapy to patient 102. An example IMD (e.g., IMD 106) of medical device system 100 may monitor one or more sensor signals from patient 102 (402). The one or more sensed signals may include sensed data (e.g., from electrodes 202, sensor(s) 206) or cardiac electrical signals. IMD 106 may determine whether IMD 106 detects an event (e.g., tachyarrhythmia) treatable by a medical therapy (e.g., ATP therapy) (404). Based on a determination that IMD 106 does not detect the event (“NO” branch of 404), IMD 106 may continue to monitor the one or more sensed signals from patient 102 (402).

If IMD 106 detects the event (“YES” branch of 404), IMD 106 may send a connection request (also referred to as “a request to connect”) to one or more external devices 108 (406). Medical device system 100 may then determine whether IMD 106 is in communication with external device 108 (408). An external device 108 is in communication with IMD 106 once processing circuitry 302 of external device 108 receives the connection request and sends a confirmation to IMD 106 which is received by processing circuitry 212 of IMD 106. The connection enables communication between IMD 106 and external device 108, e.g., to transfer information (e.g., sensed data, health metrics of patient 102, or the like), initiate delivery of ATP therapy to patient 102, modify delivery of the ATP therapy, and/or terminate delivery of the ATP therapy. If IMD 106 is not in communication with external device 108 (“NO” branch of 408), IMD 106 may return to monitoring the one or more sensed signals from patient 102 (402). IMD 106 may re-send the connection request to external device 108 e.g., after a predetermined period of time.

If IMD 106 is in communication with external device 108 (“YES” branch of 408), medical device system 100 may query external device 108 for connection with user 110 (410). External device 108 may establish the connection with user 110 via UI 208. Medical device system 100 may then determine whether external device 110 is available or accessible to user 110 (412). External device 108 may be available to user 110 if user 110 is actively interacting with external device 108, is wearing external device 108, and/or may be alerted to an occurrence of a unintended event within a short period of time.

If external device 108 is available for or accessible to user 110 (“YES” branch of 412), Medical device system 100 may deliver medical therapy (e.g., ATP therapy) to patient 102 (416). In some examples, external device 108 may instruct IMD 106 to deliver the medical therapy to patient 102. If External device 108 is not available for or accessible to user 110 (“NO” branch of 412), IMD 106 may determine whether patient 102 is experiencing an adverse event (414). The adverse event may be an event indicative that patient 102 is experiencing hemodynamic collapse. If IMD 106 determines patient 102 is experiencing an adverse event (“YES” branch of 414), medical device system 100 may deliver the medical therapy to patient 102 (416). If IMD 106 determines that patient 102 is not experiencing an adverse event, medical device system 100 may return to monitor the one or more sensed signals from patient 102 (402).

In some examples, as illustrated in FIG. 4, user 110 may be one or more persons and a connection between user 110 and external device 108 may be required to deliver medical therapy to patient 102. In other examples, such as where user 110 is a computing device, computing system, or cloud computing environment (e.g., an EMS server), after IMD 106 is in communication with external device 108 (“YES” branch of 408), medical device system 100 may deliver medical therapy to patient 102 (416) without requiring that external device 108 be available for user 110. If IMD 106 is not in communication with external device 108 (“NO” branch of 408), IMD 106 may then determine whether patient is experiencing an adverse event (414), e.g., in accordance with the example process described above.

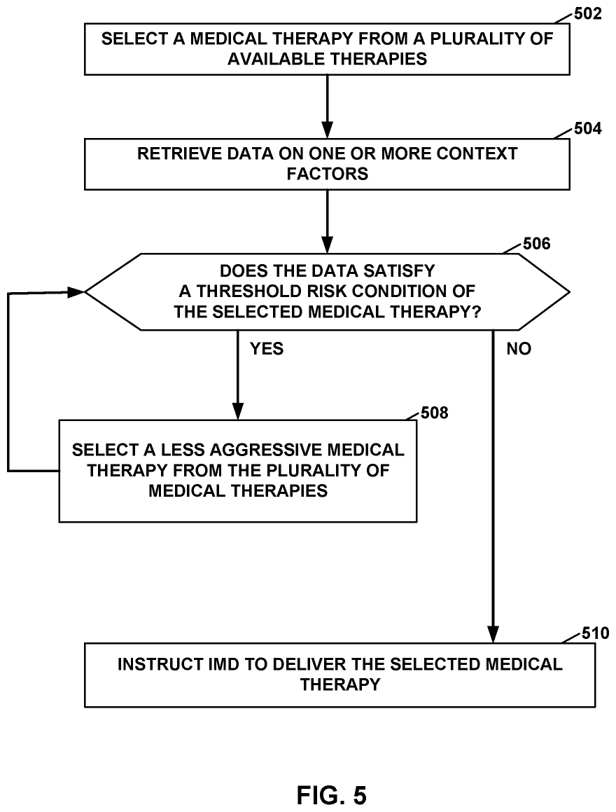

FIG. 5 is a flowchart illustrating another example process of delivering medical therapy to patient 102. Medical device system 100 may select a medical therapy (e.g., ATP therapy) from a plurality of available therapies (e.g., a plurality of ATP therapies) (502). The plurality of available therapies may be stored on external device 108 and/or network 112 of medical device system 100. Medical device system 100 may retrieve data 318 on one or more context factors (504). The context factors may include availability of patient 102 to a medical service provider, a medical care provider, and/or EMS provider, health status of patient 102, and/or behavioral patterns of patient 102. Medical device system 100 may retrieve data 318 from IMD 106 and/or one or more other computing devices, computing systems, and/or one or more cloud computing environments connected to network 112.

Medical device system 100 may determine whether data 318 satisfies a threshold risk condition of the selected medical therapy (506). The selected medical therapy may have a corresponding threshold risk condition for each of the one or more context factors. Each respective threshold risk condition may correspond to a threshold value at which the risk of an unintended event exceeds a predetermined acceptable risk. Based on a determination data 318 does not satisfy any of the threshold risk conditions (“NO” branch of 506), Medical device system 100 may proceed with the selected medical therapy and medical device system 100 may instruct IMD 106 to deliver the selected medical therapy to patient 102 (510).

If medical device system 100 determines that data 318 satisfies at least one threshold risk condition (“YES” branch of 506), medical device system 100 may select a less aggressive medical therapy (e.g., a less proarrhythmic ATP therapy) from the plurality of medical therapies (508). Medical device system 100 may then determine whether data 318 satisfies a threshold risk condition of the selected less aggressive medical therapy (506). Medical device system 100 may iteratively perform step 506 and 508 until data 318 no longer satisfies the threshold risk condition of the selected medical therapy.

The devices, systems, and techniques of this disclosure provides improvements over other medical therapy delivery systems. The establishment of a connection between external devices and implantable devices prior to deliver of medical therapies ensures availability of external aid in case of an unintended event occurrence and thereby reduces the severity of impact of any such unintended events. In some examples, using context factors to select medical therapies based on the aggressiveness of each medical therapy may increase the accuracy of tachyarrhythmia prediction, reduce the severity of impact of unintended event occurrences, and/or increase efficacy of the medical therapies while reducing risk of an unintended event.

The techniques of this disclosure may be implemented in a wide variety of computing devices, medical devices, or any combination thereof. Any of the described units, modules, or components may be implemented together or separately as discrete but interoperable logic devices. Depiction of different features as modules or units is intended to highlight different functional aspects and does not necessarily imply that such modules of units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques describes herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. For example, any module described herein may include electrical circuitry configured to perform the features attributed to that particular module, such as fixed function processing circuitry, programmable processing circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

It should be noted that medical device system 100, and the techniques described herein, may not be limited to use in a human patient. In alternative examples, medical device system 100 may be implemented in non-human patients, e.g., primates, canines, equines, pigs, and felines. These other animals may undergo clinical or research therapies that my benefit from the subject matter of this disclosure. Various examples are described herein, such as the following examples.

### Example 1

an implantable medical device (IMD) configured to deliver anti-tachycardia pacing (ATP) therapy to a patient, the IMD comprising: therapy delivery circuitry connected to one or more electrodes; communications circuitry; and processing circuitry configured to: transmit, via the communications circuitry, a request to connect to an external device; determine, based on received signals from the external device, whether the IMD is in communication with the external device; and based on a determination that the IMD is in communication with the external device, transmit instructions to the therapy delivery circuitry to deliver the ATP therapy to the patient.

### Example 2

the IMD of example 1, wherein the IMD is implanted within a heart of the patient, and wherein the therapy delivery circuitry is configured to deliver the ATP therapy to tissue or wall tissue of one or more chambers of the heart of the patient.

### Example 3

the IMD of any of examples 1 and 2, wherein to determine whether the IMD is in communication with the external device, the processing circuitry is configured to: determine, based on the received signals, whether the external device is accessible to a user; and based on a determination that the external device is accessible to the user, determine that the IMD is in communication with the external device.

### Example 4

the IMD of any of examples 1-3, wherein the processing circuitry is further configured to: based on a determination that the IMD is not in communication with the external device, transmit a second request to connect to the external device after a predetermined period of time.

### Example 5

the IMD of any of examples 1-4, further comprising sensing circuitry, and wherein the processing circuitry is further configured to: determine, based on the received data, that the IMD is not in communication with the external device; determine, based on sensed data from the sensing circuitry, whether the patient is experiencing an adverse event indicative of hemodynamic collapse; and based on a determination that the patient is experiencing the adverse event, transmit instructions to the therapy delivery circuitry to deliver the ATP therapy to the patient via the one or more electrodes.

### Example 6

the IMD of example 5, wherein the adverse event comprises one or more of a fall of the patient, a cessation of breathing by the patient, a motion of the heart of the patient, a drop in tissue oxygenation of the patient, or a tachycardiac event experienced by the patient.

### Example 7

a system comprising: an implantable medical device (IMD) configured to deliver an anti-tachycardia pacing (ATP) therapy to a patient; and an external device comprising: communications circuitry configured to communicate with the IMD; and processing circuitry configured to: receive, via the communications circuitry, a request to connect from the IMD; determine whether the IMD is in communication with the external device; and based on a determination that the IMD is in communication with the external device, transmit instructions, via the communications circuitry, to the IMD to deliver the ATP therapy to the patient.

### Example 8

a system of example 7, wherein the IMD comprises an implantable cardiac device, and wherein the IMD is configured to, upon receiving the instructions to deliver the ATP therapy to the patient, deliver the ATP therapy to tissue or wall tissue of one or more chambers of a heart of the patient.

### Example 9

a system of any of examples 7 and 8, wherein to transmit the instructions to the IMD, the processing circuitry is configured to: determine whether the external device is accessible to a user; and transmit the instructions to the IMD based on a determination that the external device is accessible to the user.

### Example 10

the system of example 9, wherein the external device further comprises a user interface (UI), and wherein to determine whether the external device is accessible to the user, the processing circuitry is configured to determine whether the UI received user input from the user.

### Example 11

the system of example 10, wherein the user input comprises one or more of a tactile input, a visual input, or an auditory input.

### Example 12

the system of any of examples 9-11, wherein the user comprises one or more of a medical service provider or emergency medical services.

### Example 13

the system of any of examples 7-12, wherein the IMD is configured, without receiving the instructions, to transmit the request to connect to the external device after a predetermined period of time.

### Example 14

the system of any of examples 7-13, wherein the IMD is configured to: determine that the IMD is not in communication with the external device; determine, based on data sensed by the IMD, whether the patient is experiencing an adverse event indicative of hemodynamic collapse; and based on a determination that the patient is experiencing the adverse event, deliver the ATP therapy to the patient.

### Example 15

the system of example 14, wherein the adverse event comprises one or more of a fall of the patient, a cessation of breathing by the patient, a motion of a heart of the patient, a drop in tissue oxygenation of the patient, or a tachycardiac event experienced by the patient.

### Example 16

a system comprising: an external device configured to communicate with a network; and an implantable medical device (IMD) comprising: memory configured to store a plurality of medical therapies, wherein each of the plurality of medical therapies is configured to be delivered by the IMD to a patient; communications circuitry configured to communicate with the external device; signal generation circuitry; and processing circuitry configured to: select a first medical therapy from the plurality of medical therapies; receive, from the external device and via the communications circuitry, data on one or more context factors for the patient; determine, based on the received data, whether the received data satisfies one or more threshold risk conditions of the patient; select, based on a determination that the received data satisfies at least one of the one or more threshold risk conditions of the first medical therapy, a second medical therapy from the plurality of medical therapies, wherein the second medical therapy is less aggressive than the first medical therapy; and deliver, via the signal generation circuitry, the second medical therapy to the patient.

### Example 17

the system of example 16, wherein the IMD comprises an implantable cardiac device, and wherein delivering the second medical therapy to the patient comprises delivering the second medical therapy to tissue or wall tissue of one or more chambers of a heart of the patient.

### Example 18

the system of example 17, wherein the second medical therapy is less proarrhythmic than the first medical therapy.

### Example 19

the system of any of examples 16-18, wherein the first medical therapy comprises a first anti-tachycardiac pacing (ATP) therapy wherein the second medical therapy comprises a second ATP therapy, each of the first ATP therapy and the second ATP therapy comprising a corresponding set of therapy parameters.

### Example 20

the system of example 19, wherein each corresponding set of therapy parameters comprises a maximum therapy amplitude, a therapy delivery frequency, and a number of safety checks.

### Example 21

the system of example 20, wherein the corresponding set of therapy parameters for the first ATP therapy comprises one or more of a higher maximum therapy amplitude, a higher therapy delivery frequency, or a smaller number of safety checks relative to the corresponding set of therapy parameters for the second ATP therapy.

### Example 22

the system of any of examples 16-21, wherein the external device is configured to: receive a request to connect from the IMD; determine whether the IMD is in communication with the external device; and transmit instructions to deliver the second medical therapy to the patient to the IMD based on a determination that the IMD is in communication with the external device.

### Example 23

the system of any of examples 16-22, wherein the one or more context factors comprises one or more of a proximity of a medical service provider to the patient, a responsiveness of the medical service provider to the patient, a proximity of emergency medical services, a health status of the patient, or behavioral patterns of the patient.

### Example 24

the system of example 23, wherein the one or more threshold conditions comprises one or more of a threshold distance between the medical service provider and the patient or a threshold response time of the medical service provider to a medical event experienced by the patient.

### Example 25

the system of example 24, wherein the medical event comprises an arrhythmia.

### Example 26

the system of any of examples 23-25, wherein the one or more threshold conditions comprises a threshold distance between the emergency medical services and the patient.

### Example 27

the system of any of examples 16-26, wherein the processing circuitry is configured to, based on a determination that the received data does not satisfy any of the one or more threshold conditions, select a third medical therapy from the plurality of medical therapies, wherein the third medical therapy is more aggressive than the first medical therapy.

### Example 28

the system of any of examples 16-27, wherein the third medical therapy is more proarrhythmic than the first medical therapy.

### Example 29

the system of any of examples 27 and 28, wherein the IMD is configured to perform fewer safety checks before administration of the third medical therapy relative to the first medical therapy.

### Example 30

the system of any of examples 27-29, wherein the third medical therapy includes instructions to deliver electrical stimulation signals to the patient at a higher frequency relative to the first medical therapy.

### Example 31

a method comprising: transmitting, by communications circuitry of an implantable medical device (IMD), a request to connect to an external device; receiving, by the communications circuitry, signals from the external device.

### Example 32

the method of example 31, wherein the IMD is implanted within a heart of the patient, and wherein transmitting delivering ATP therapy to the patient comprises: delivering, by the therapy delivery circuitry, the ATP therapy to tissue or wall tissue of one or more chambers of the heart.

### Example 33

the method of any of examples 31 and 32, wherein determining whether the IMD is in communication with the external device comprises: determining, by the processing circuitry and based on the received signals, whether the external device is accessible to a user; and based on a determination that the external device is accessible to the user, determining, by the processing circuitry, that the IMD is in communication with the external device.

### Example 34

the method of any of examples 31-33, further comprising: based on a determination that the IMD is not in communication with the external device, transmitting, by the communications circuitry, a second request to connect to the external device after a predetermined period of time.

### Example 35

the method of any of examples 31-34, further comprising: determining, by the processing circuitry and based on the received data, that the IMD is not in communication with the external device; sensing, by sensing circuitry of the IMD, data from the patient; determining, by the processing circuitry and based on the sensed data, whether the patient is experiencing an adverse event indicative of hemodynamic collapse; and based on a determination that the patient is experiencing the adverse event, delivering, by the therapy delivery circuitry, the ATP therapy to the patient.

### Example 36

the method of example 35, wherein the adverse event comprises one or more of a fall of the patient, a cessation of breathing by the patient, a motion of a heart of the patient, a drop in tissue oxygenation of the patient, or a tachycardiac event experienced by the patient.

### Example 37

a method comprising: receiving, by processing circuitry of an external device, a request to connect from an implantable medical device (IMD); determining, by the processing circuitry, whether the IMD is in communication with the external device; and transmitting, by the processing circuitry and based on a determination that the IMD is in communication with the external device, instructions to the IMD to deliver an anti-tachycardia pacing (ATP) therapy to a patient.

### Example 38

the method of example 37, wherein the IMD comprises an implantable cardiac device, and wherein the method further comprises: delivering, by the IMD and in response to receiving the instructions to deliver the ATP therapy to the patient, the ATP therapy to tissue or wall tissue of one or more chambers of a heart of the patient.

### Example 39

the method of any of examples 37-38, wherein transmitting the instructions to the IMD comprises: determining, by the processing circuitry, whether the external device is accessible to a user; and transmitting, by the processing circuitry, the instructions to the IMD based on a determination that the external device is accessible to the user.

### Example 40

the method of example 39, wherein the external device comprises a user interface (UI), and wherein determining whether the external device is in communication with the user comprises: determining, by the processing circuitry, whether the UI received user input from the user.

### Example 41

the method of example 40, wherein the user input comprises one or more of a tactile input, a visual input, or an auditory input.

### Example 42

the method of any of examples 39-41, wherein the user comprises one or more of a medical service provider or emergency medical services.

### Example 43

the method of any of examples 37-42, further comprising transmitting, by the IMD without receiving the instructions, the request to connect to the external device after a predetermined period of time.

### Example 44

the method of any of examples 37-43, further comprising: determining, by the processing circuitry, that the IMD is not in communication with the external device; determining, based on data sensed by the IMD, whether the patient is experiencing an adverse event indicative of hemodynamic collapse; and based on a determination that the patient is experiencing the adverse event, transmitting, by the processing circuitry, instructions to the IMD to deliver the ATP therapy to the patient.

### Example 45

the method of example 44, wherein the adverse event comprises one or more of a fall of the patient, a termination of breathing by the patient, a motion of a heart of the patient, a drop in tissue oxygenation of the patient, or a tachycardiac event experienced by the patient.

### Example 46

a method comprising: selecting, by processing circuitry of an IMD, a first medical therapy from a plurality of medical therapies, wherein each of the plurality of medical therapies is configured to be delivered by the IMD to a patient; receiving, from a network and via an external device, data on one or more context factors for the patient; determining, by the processing circuitry and based on the received data, whether the received data satisfies one or more threshold risk conditions of the patient; selecting, by the processing circuitry and based on a determination that the received data satisfies at least one of the one or more threshold risk conditions of the first medical therapy, a second medical therapy from the plurality of medical therapies, wherein the second medical therapy is less aggressive than the first medical therapy; and delivering, by signal generation circuitry of the IMD, the second medical therapy to the patient.

### Example 47

the method of example 46, wherein the IMD comprises an implantable cardiac device, and wherein delivering the second medical therapy to the patient comprises: delivering, by the signal generation circuitry, the second medical therapy to tissue or wall tissue of one or more chambers of a heart of the patient.

### Example 48

the method of example 47, wherein the second medical therapy is less proarrhythmic than the first medical therapy.

### Example 49

the method of any of examples 47 and 48, wherein the first medical therapy comprises a first anti-tachycardia pacing (ATP) therapy wherein the second medical therapy comprises a second ATP therapy, each of the first ATP therapy and the second ATP therapy comprising a corresponding set of therapy parameters.

### Example 50

the method of example 49, wherein each corresponding set of therapy parameters comprises a maximum therapy amplitude, a therapy delivery frequency, and a number of safety checks.

### Example 51

the method of example 50, wherein the corresponding set of therapy parameters for the first ATP therapy comprises one or more of a higher maximum therapy amplitude, a higher therapy delivery frequency, or a smaller number of safety checks relative to the corresponding set of therapy parameters for the second ATP therapy.

### Example 52

the method of any of examples 46-51, further comprising: delivering, by the processing circuitry and via the communications circuitry, a request to connect to the external device; determining, by the processing circuitry, whether the IMD is in communication with the external device; and transmitting, by the processing circuitry and based on a determination that the IMD is in communication with the external device, instructions to the signal generation circuitry to deliver the second medical therapy.

### Example 53

the method of any of examples 46-52, wherein the one or more context factors comprises one or more of a proximity of a medical service provider to the patient, a responsiveness of the medical service provider to the patient, a proximity of emergency medical services, a health status of the patient, or behavioral patterns of the patient.

### Example 54

the method of example 53, wherein the one or more threshold conditions comprises one or more of a threshold distance between the medical service provider and the patient or a threshold response time of the medical service provider to a medical event experienced by the patient.

### Example 55

the method of example 54, wherein the medical event comprises an arrhythmia.

### Example 56

the method of any of examples 53-55, wherein the one or more threshold conditions comprises a threshold distance between the emergency medical services and the patient.

### Example 57

the method of any of examples 46-56, wherein the second medical therapy comprises instructions to the IMD to perform an increased number of safety checks before administration of the second medical therapy relative to the first medical therapy.

### Example 58

the method of any of examples 46-57, wherein the second medical therapy comprises instructions to the IMD to deliver electrical stimulation signals to the patient at a lower frequency relative to the first medical therapy.

### Example 59

the method of any of claims 46-58, further comprising: selecting, by the processing circuitry and based on a determination that the received data does not satisfy any of the one or more threshold conditions, a third medical therapy from the plurality of medical therapies, wherein the third medical therapy is more aggressive than the first medical therapy.

### Example 60

the method of any of examples 46-59, wherein the third medical therapy is more proarrhythmic than the first medical therapy.

### Example 61

the method of any of examples 59 and 60, wherein the third medical therapy comprises instructions to the IMD to perform a reduced number of safety checks before administration of the third medical therapy relative to the first medical therapy.

### Example 62

the method of any of examples 59-61, wherein the third medical therapy comprises instructions to deliver electrical stimulation signals to the patient at a higher frequency relative to the first medical therapy.

### Example 63

a computer readable storage medium comprising instructions that, when executed, cause processing circuitry within a system to perform the method of any of examples 31-36.

### Example 64

a computer readable storage medium comprising instructions that, when executed, cause processing circuitry within a system to perform the method of any of examples 37-45.

### Example 65

a computer readable storage medium comprising instructions that, when executed, cause processing circuitry within a system to perform the method of any of examples 46-62.

### Example 66

a system comprising an IMD of any of examples 1-6, and an external device of any of examples 7-15.

1: An implantable medical device (IMD) configured to deliver anti-tachycardia pacing (ATP) therapy to a patient, the IMD comprising:
therapy delivery circuitry connected to one or more electrodes;
communications circuitry; and
processing circuitry configured to:
transmit, via the communications circuitry, a request to connect to an external device;
determine, based on received signals from the external device, whether the IMD is in communication with the external device; and
based on a determination that the IMD is in communication with the external device, transmit instructions to the therapy delivery circuitry to deliver the ATP therapy to the patient.

2: The IMD of claim 1, wherein the IMD is implanted within a heart of the patient, and wherein the therapy delivery circuitry is configured to deliver the ATP therapy to tissue of one or more chambers of the heart of the patient.

3: The IMD of claim 1, wherein to determine whether the IMD is in communication with the external device, the processing circuitry is configured to:
determine, based on the received signals, whether the external device is accessible to a user; and
based on a determination that the external device is accessible to the user, determine that the IMD is in communication with the external device.

4: The IMD of claim 1, wherein the processing circuitry is further configured to:
based on a determination that the IMD is not in communication with the external device, transmit a second request to connect to the external device after a predetermined period of time.

5: The IMD of claim 1, further comprising sensing circuitry, and wherein the processing circuitry is further configured to:
determine, based on the received data, that the IMD is not in communication with the external device;
determine, based on sensed data from the sensing circuitry, whether the patient is experiencing an adverse event indicative of hemodynamic collapse; and
based on a determination that the patient is experiencing the adverse event, transmit instructions to the therapy delivery circuitry to deliver the ATP therapy to the patient via the one or more electrodes.

6: The IMD of claim 5, wherein the adverse event comprises one or more of a fall of the patient, a cessation of breathing by the patient, a motion of the heart of the patient, a drop in tissue oxygenation of the patient, or a tachycardiac event experienced by the patient.

7: A system comprising:
an implantable medical device (IMD) configured to deliver an anti-tachycardia pacing (ATP) therapy to a patient; and
an external device comprising:
communications circuitry configured to communicate with the IMD; and
processing circuitry configured to:
receive, via the communications circuitry, a request to connect from the IMD;
determine whether the IMD is in communication with the external device; and
based on a determination that the IMD is in communication with the external device, transmit instructions, via the communications circuitry, to the IMD to deliver the ATP therapy to the patient.

8: The system of claim 7, wherein the IMD comprises an implantable cardiac device, and wherein the IMD is configured to, upon receiving the instructions to deliver the ATP therapy to the patient, deliver the ATP therapy to tissue of one or more chambers of a heart of the patient.

9: The system of claim 7, wherein to transmit the instructions to the IMD, the processing circuitry is further configured to:
determine whether the external device is accessible to a user; and
transmit the instructions to the IMD based on a determination that the external device is accessible to the user.

10: The system of claim 9, wherein the external device further comprises a user interface (UI), and wherein to determine whether the external device is accessible to the user, the processing circuitry is configured to determine whether the UI received user input from the user.

11: The system of claim 10, wherein the user input comprises one or more of a tactile input, a visual input, or an auditory input.

12: The system of claim 9, wherein the user comprises one or more of a medical service provider or emergency medical services.

13: The system of claim 7, wherein the IMD is configured, without receiving the instructions, to transmit the request to connect to the external device after a predetermined period of time.

14: The system of claim 7, wherein the IMD is configured to:
determine that the IMD is not in communication with the external device;
determine, based on data sensed by the IMD, whether the patient is experiencing an adverse event indicative of hemodynamic collapse; and
based on a determination that the patient is experiencing the adverse event, deliver the ATP therapy to the patient.

15: The system of claim 14, wherein the adverse event comprises one or more of a fall of the patient, a cessation of breathing by the patient, a motion of a heart of the patient, a drop in tissue oxygenation of the patient, or a tachycardiac event experienced by the patient.

16: A system comprising:
an external device configured to communicate with a network; and
an implantable medical device (IMD) comprising:
memory configured to store a plurality of medical therapies, wherein each of the plurality of medical therapies is configured to be delivered by the IMD to a patient;
communications circuitry configured to communicate with the external device;
signal generation circuitry; and
processing circuitry configured to:
select a first medical therapy from the plurality of medical therapies;
receive, from the external device and via the communications circuitry, data on one or more context factors for the patient;
determine, based on the received data, whether the received data satisfies one or more threshold risk conditions of the patient;
select, based on a determination that the received data satisfies at least one of the one or more threshold risk conditions of the first medical therapy, a second medical therapy from the plurality of medical therapies, wherein the second medical therapy is less aggressive than the first medical therapy; and
deliver, via the signal generation circuitry, the second medical therapy to the patient.

17: The system of claim 16, wherein the IMD comprises an implantable cardiac device, and wherein delivering the second medical therapy to the patient comprises delivering the second medical therapy to wall tissue of one or more chambers of a heart of the patient.

18: The system of claim 17, wherein the second medical therapy is less proarrhythmic than the first medical therapy.

19: The system of any of claims 17 and 18, wherein the first medical therapy comprises a first anti-tachycardia pacing (ATP) therapy wherein the second medical therapy comprises a second ATP therapy, each of the first ATP therapy and the second ATP therapy comprising a corresponding set of therapy parameters, and wherein each corresponding set of therapy parameters comprises a maximum therapy amplitude, a therapy delivery frequency, and a number of safety checks.

20: The system of claim 19, wherein the corresponding set of therapy parameters for the first ATP therapy comprises one or more of a higher maximum therapy amplitude, a higher therapy delivery frequency, or a smaller number of safety checks relative to the corresponding set of therapy parameters for the second ATP therapy.
